(19) United States
(12) Patent Application Publication
(10) Pub. No.: US 2010/0016664 A1
(43) Pub. Date: Jan. 21, 2010

(54) SURGICAL VISUAL OBTURATOR

(75) Inventor: Frank J. Viola, Sandy Hook, CT (US)

Correspondence Address: Tyco Healthcare Group LP, 60 MIDDLETOWN AVENUE, NORTH HAVEN, CT, 06473, US

(73) Assignee: TYCO HEALTHCARE GROUP LP, North Haven, CT (US)

(21) Appl. No.: 12/513,459

(22) PCT Filed: Dec. 20, 2007
(86) PCT No.: PCT/US2007/026239

§ 371 Date: May 4, 2009

Related U.S. Application Data
(60) Provisional application No. 60/875,891, filed on Dec. 20, 2006.

(51) Int. Cl. A61B 1/00; A61B 1/06

(52) U.S. Cl. 600/114; 600/160

(57) ABSTRACT

An obturator apparatus for penetration of tissue and for permitting visualization includes an elongated shaft having proximal and distal ends, and defining a longitudinal axis and an optical transmitting member mounted adjacent the distal end of the elongated shaft. The optical transmitting member comprises a transparent material for permitting passage of light therethrough and has internal surfaces defining a hollow internal chamber and external surfaces adapted to penetrate tissue. The optical transmitting member is further adapted to provide an inclined field of view inclined relative to the longitudinal axis. The elongated shaft may include a longitudinal opening adapted for reception of an endoscope.

### BACKGROUND

1. Technical Field

The present disclosure relates to an apparatus for penetration of body tissue and particularly relates to an obturator apparatus having penetration and visualization capabilities.

2. Background of the Related Art

Endoscopic surgical procedures, that is, surgical procedures performed through tubular sleeves or cannulas, have been utilized for many years. Initially, endoscopic surgical procedures were primarily diagnostic in nature. More recently as endoscopic technology has advanced, surgeons are performing increasingly complex and innovative endoscopic surgical procedures. In endoscopic procedures, surgery is performed in any hollow viscus of the body through a small incision or through narrow endoscopic tubes (cannulas) inserted through small entrance wounds in the skin. In laparascopic procedures, surgery is performed in the interior of the abdomen.

Typically, after the surgical region is insufflated, trocars are used to puncture the body cavity. These trocars include a cannula which remains in place for use during endoscopic procedures. Generally, trocars used during such procedures include a stylet having a sharp tip for penetrating the body cavity positioned coaxially within protective tubes to protect a patient or surgeon from inadvertent contact with the tip.

In the past, surgeons have had to blindly insert the trocar assembly into the body cavity without knowing whether to alter the path of the penetrating member to avoid blood vessels and organs. The optical penetrating trocar of the present invention provides a means for the surgeon to fully visualize the operation before, during, and after penetration, therefore preventing unnecessary complications.

### SUMMARY

Accordingly, the present disclosure is directed to further improvements in penetrating tissue during surgical procedures such as endoscopic or laparoscopic surgery. In particular, an obturator apparatus for penetration of tissue and for permitting visualization includes an elongated shaft having proximal and distal ends, and defining a longitudinal axis and an optical transmitting member mounted adjacent the distal end of the elongated shaft. The optical transmitting member comprises a transparent material for permitting passage of light therethrough and has internal surfaces defining a hollow internal chamber and external surfaces adapted to penetrate tissue. The optical transmitting member is further adapted to provide an inclined field of view inclined relative to the longitudinal axis. The elongated shaft may include a longitudinal opening adapted for reception of an endoscope.

At least one of the internal and external surfaces of the optical transmitting member is a refracting surface adapted to direct the light along a predetermined path. The optical transmitting member may be adapted to direct light in general parallel direction with the inclined field of view, or, alternatively, adapted to direct light in general parallel relation to the longitudinal axis of the elongated shaft.

The optical transmitting member may define a general pyramidal configuration, a general conical configuration, a general jeweled configuration or may include a fresnel lens.

A piercing member may be mounted to the optical transmitting member for piercing tissue. Preferably, the piercing member is coaxially mounted to the optical transmitting member and defines a piercing end extending from the optical transmitting member.

The obturator apparatus may incorporate illumination means for providing illuminating light and imaging means for transferring an image of an object.

In another embodiment, the obturator apparatus includes an elongated shaft defining proximal and distal ends, and defining a longitudinal axis, a transparent member adjacent the distal end thereof and having a refracting surface adapted to provide an inclined angle of view, and a piercing member mounted to the transparent member and dimensioned to pierce tissue. The piercing member is mounted to the transparent member in coaxial relation therewith.

In a further alternate embodiment, an optical obturator apparatus for penetration of tissue and for permitting visualization of a surgical object with an endoscope coupled to the obturator apparatus is provided. The endoscope includes an illumination system for delivering illuminating light and an imaging system for detecting and transmitting an illuminated image of the surgical object. The obturator apparatus includes an elongated shaft adapted to at least partially receive the endoscope, and having proximal and distal ends and an optical transmitting member mounted adjacent the distal end of the elongated shaft. The optical transmitting member is adapted to penetrate tissue and defines a hollow interior chamber. The optical transmitting members includes a transparent material whereby light rays projected by the illumination system of the inserted endoscope passes through the internal chamber for emission outwardly by the optical transmitting member to permit viewing of the surgical object through the optical transmitting member with the imaging system of the endoscope. The optical transmitting member is adapted to provide a field of view angularly offset relative to the longitudinal axis.

Other features and advantages of the present disclosure will become apparent from the following description of preferred embodiments of the disclosure with reference to the accompanying drawings.

### BRIEF DESCRIPTION OF THE DRAWINGS

Preferred embodiments of the present disclosure are described hereinbelow with references to the drawings, wherein:

FIG. 1 is a perspective view of the obturator apparatus in accordance with the principles of the present disclosure and depicted with an endoscope;

FIG. 2 is an enlarged partial cross-sectional view of an embodiment of the optical transmitting member of the obturator apparatus;

FIG. 3 is an enlarged partial cross-sectional view of another alternate embodiment of the obturator apparatus;

FIG. 4 is an enlarged partial cross-sectional view of another alternate embodiment of the obturator apparatus;

FIG. 5 is an enlarged partial cross-sectional view of another alternate embodiment of the obturator apparatus;

FIG. 6 is an enlarged partial cross-sectional view of another alternate embodiment of the obturator apparatus;

FIG. 7 is an enlarged partial cross-sectional view of another alternate embodiment of the obturator apparatus;

FIG. 8 is an enlarged partial cross-sectional view of another alternate embodiment of the obturator apparatus; and

FIG. 9 is an enlarged partial cross-sectional view of another alternate embodiment of the obturator apparatus.

### DETAILED DESCRIPTION OF THE PREFERRED EMBODIMENTS

The apparatus of the present disclosure is provided to penetrate body tissue during endoscopic and laparoscopic surgical procedures and to provide a simultaneous generally forward directional view of the body tissue being penetrated. In one embodiment, the apparatus includes a trocar and cannula assembly similar to that found in U.S. Pat. No. 5,658,236, the entire contents of which is incorporated herein by reference. The present disclosure describes an obturator apparatus which provides a viable option to surgeons who are unable to clearly visualize the interior of the body cavity.

In the following description, as is traditional, the term “proximal” refers to the portion of the apparatus closest to the operator, while the term “distal” refers to the portion of the apparatus remote from the operator.

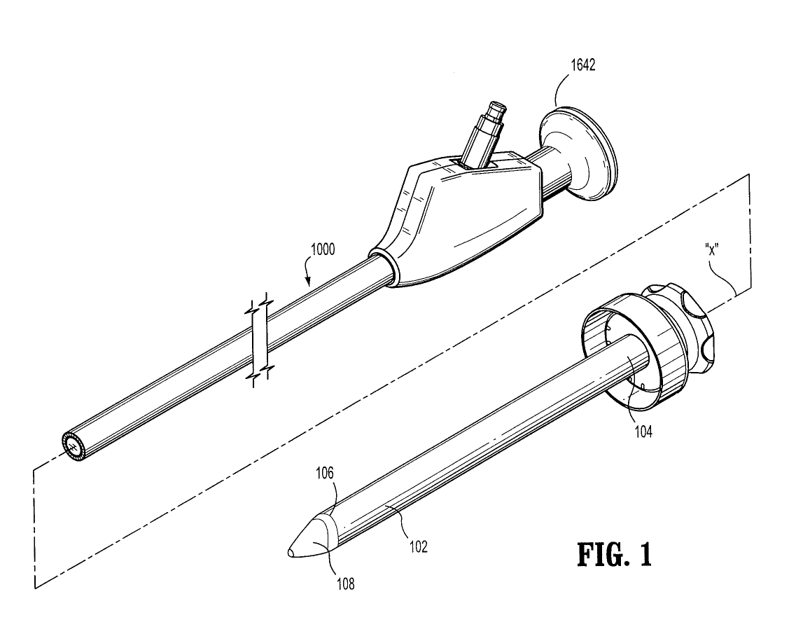

Referring now to the drawings, in which like reference numerals identify identical or substantially similar parts throughout the several views, FIG. 1 illustrates, in perspective view, the obturator apparatus 100 of the present disclosure. Obturator assembly 100 may be used with or without an endoscope assembly 1000. In one alternative, obturator assembly 100 incorporates its own illumination and imaging means. Obturator apparatus 100 is configured to penetrate tissue and permit visualization of the tissue to the surgeon performing the procedure. Obturator apparatus 100 may be constructed of a plurality of different materials including but not limited to stainless steel, titanium and/or alloys thereof, polymeric materials, and ceramics. Obturator apparatus 100 may be disposable after use or reusable. If reusable, obturator apparatus 100 may be sterilized for subsequent use.

Endoscope 1000 may be any conventional scope suitable for endoscopic applications including, e.g., a laparoscope, arthroscope, colonoscope, etc. In one preferred embodiment, endoscope 1000 may be the scope disclosed in commonly assigned U.S. Pat. No. 5,412,504 to Leiner, the entire contents of which disclosure are hereby incorporated by reference. Endoscope 1000 incorporates an optical train or lens arrangement which is capable of transmitting an image of an object from the distal or objective lens through the eyepiece or monitor for viewing by the surgeon. Further details of endoscope 1000 may be ascertained by reference to the '504 patent.

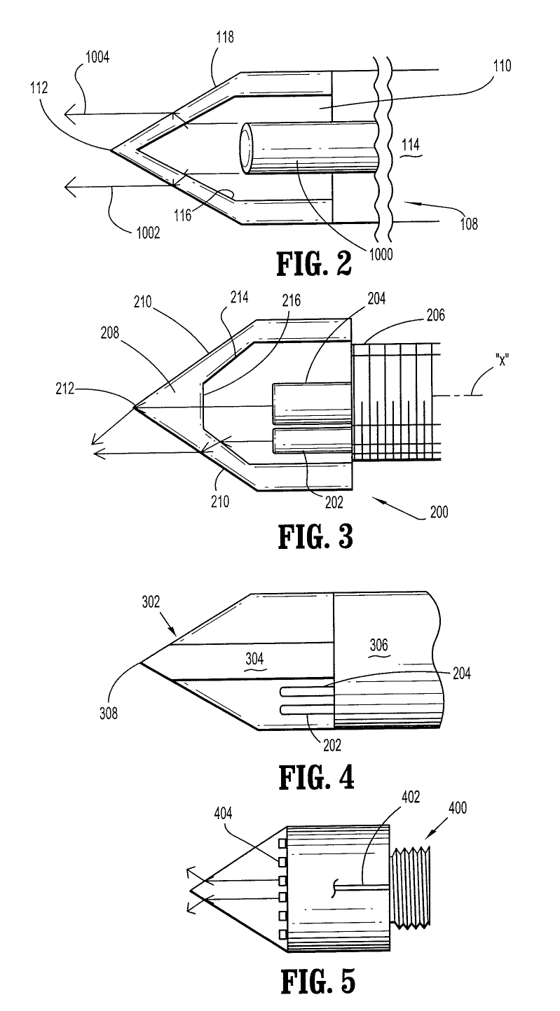

Referring to FIGS. 1-2, obturator apparatus 100 includes elongated shaft 102 having proximal and distal ends 104, 106, and defining a longitudinal axis “x”. Optionally, elongated shaft 102 may be substantially hollow in structure to provide a passageway for lighting and optical devices as will be discussed hereinafter. Shaft 102 may have optical transmitting member 108 which is situated adjacent distal end 106 of shaft 102. Optical transmitting member 108 is adapted to penetrate tissue and defines hollow interior chamber 110. Optical transmitting member 108 may include penetrating tip 112 and securing end 114. Penetrating tip 112 may be sharp to pierce tissue or alternatively may be blunt to provide for tissue dissection and/or separation. Securing end 114 may be attached to shaft 102 by means of a threaded design or any other suitable design. Optical transmitting member 108 may be either permanently secured to distal end 106 of shaft 102 or be removable. Optical transmitting member 108 may be constructed from a number of transparent materials including, but not limited to glass, acrylic glass, polystyrene, or polycarbonate. Optical transmitting member 108 may be generally conically-shaped or pyramidal in configuration.

Optical transmitting member 108 may have an interior surface 116 and an exterior surface 118. Both interior surface 116 and exterior surface 118 may be refracting surfaces which are adapted to direct light along a predetermined path. Interior surface 116 defines hollow interior chamber 110. Interior surface 116 of optical transmitting member 108 may be arranged in a variety of different configurations. Some of these may include concave, convex, straight surfaces or lenses and a fresnel lenses. In one preferred embodiment, interior and exterior surfaces 116,118 are refracting surfaces adapted to receive and/or transmit light rays along a direction substantial in parallel relation to axis “x” as depicted in FIG. 2. This arrangement provides for generally direct or forward viewing and illumination of the surgical site. In FIG. 2, endoscope 1000 is shown positioned within elongated shaft 102 with the distal end of the endoscope 1000 disposed within hollow internal chamber 110 of optical transmitting member. For exemplative purposes, light rays 1002, 1004 are representative of the light transmitted or received by illumination and optical systems of the endoscope 1000. In the alternative, interior and exterior surfaces 116, 118 may be adapted to provide for inclined transmission or reception of light rays, i.e., inclined relative to the longitudinal axis of the elongated shaft 102. Exterior surface 118 is adapted to pass through tissue and provide the means for entering the body.

With reference now to FIG. 3, in an alternate embodiment, obturator apparatus 200 incorporates its own illumination and optical devices or systems. For example, obturator apparatus includes illumination means 202 and image transmitting means 204 each extending in a general longitudinal direction through the shaft 206 and terminating within optical transmitting member 208. Illumination means 202 is configured to transfer illuminating light to the operative site. Optionally, illumination means 202 may be adjustably positionable within the shaft 206 in an axial direction, and/or in a direction transverse to the longitudinal axis, in order to selectively direct light to the surgical area. Illumination means 202 may include fiber optics or a liquid light transferring medium. Illumination means 204 may include a bundle of fiber optic elements or lenses which transfer an optical image for viewing by the surgeon.

Optical transmitting member 208 includes external surfaces 210 extending to pointed end 212 and internal surfaces 214, 216. Internal surfaces 214 are inclined relative to the axis “x” of shaft 206. Internal surface 216 is generally transverse to the axis “x”. It is envisioned that external surfaces 210 and internal surfaces 214, 216 may be arranged to provide an inclined angle of view for image transmitting means 204 as depicted by the light ray arrow “i” while illumination means 202 includes a generally forward light directional vector. In the alternative, the illumination means 202 may be adapted to also direct light at an angle relative to the axis “x”.

Referring now to FIG. 4, an alternate embodiment of the obturator apparatus 300 is disclosed. In accordance with this embodiment, the optical transmitting member 302 may have a metal piercing member 304 extending through the transmitting member 302 in, e.g., coaxial arrangement therewith. Piercing member 304 may extend through shaft 306 of the apparatus 300. Alternatively, piercing member 304 could be affixed to optical transmitting member 302 using a variety of different arrangements, including standard adhesive techniques well known in the art. Moreover, piercing member 304 could be constructed of a plurality of different materials including but not limited to stainless steel, titanium, and/or alloys thereof or alternatively of a suitable polymeric material. It is also envisioned that piercing member 304 may be movable relative to optical transmitting member 302 between a retracted position where the piercing tip 308 is enclosed within the transmitting member 302 and an advanced position as shown in FIG. 3 where the piercing tip 308 is exposed. Light transmitting member 302 functions to transmit light for illumination or imaging in the manner discussed hereinabove.

Referring now to FIG. 5, an alternate embodiment of the obturator apparatus 400 is disclosed. In accordance with this embodiment, the illumination means 402 contained within the apparatus 400 may consist of a string of light emitting diodes (LEDs) 404. LEDs 404 are capable of emitting light of an intended color without the use of color filters that traditional lighting methods require. Moreover, LEDs 404 provide an extremely long life span and require minimal maintenance. Various configurations of LEDs 404 may be used to produce the desired illumination. Utilizing different shapes and numbers of LED's 404 may allow light to be focused in a variety of different arrangements.

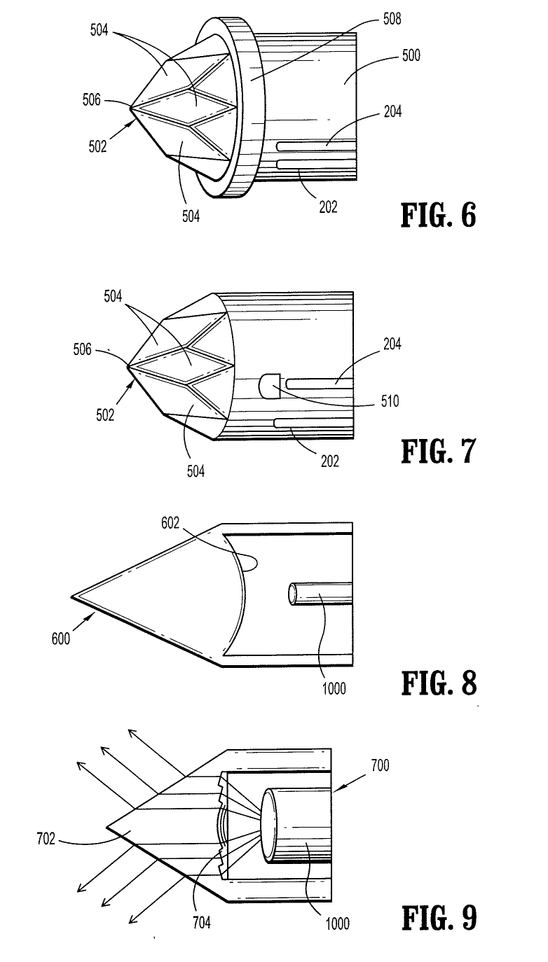

FIG. 6 illustrates an alternate embodiment of the obturator apparatus 500. In accordance with this embodiment, the optical transmitting member 502 may have a jeweled or diamond-shaped piercing appearance having a plurality of intersecting surfaces 504 and piercing tip 506. Optical transmitting member 502 may be constructed of a rigid transparent material such as glass, acrylic glass, polystyrene, or polycarbonate. In addition, obturator apparatus 500 incorporates optics lens 508 which is in optical communication with image transmitting means 204 to receive and transfer the image. Lens 508 may be mounted or integrally formed about the periphery of transmitting member 502. This configuration may utilize similar lighting and imaging methods as referenced above.

FIG. 7 illustrates an alternate embodiment of FIG. 6. In accordance with this embodiment, optical transmitting member 502 is devoid of optics lens 508, and serves both to transfer illumination and the image. Preferably, with this embodiment, apparatus 500 incorporates correcting lens 510 which serves to correct the image received via optical transmitting member 502. A suitable correcting lens 510 adapted to achieve this purpose would be readily appreciated by one skilled in the art.

FIG. 8 illustrates a further alternate embodiment of the obturator apparatus. FIG. 8 discloses obturator apparatus 600 wherein interior surface 602 is generally convex. Endoscope 1000 is shown positioned within apparatus 600 and replaces the built in illumination and image transmitting means 202, 204.

Referring now to FIG. 9, an alternate embodiment of obturator apparatus 700 is disclosed. In this embodiment, optical transmitting member 702 incorporates fresnel lens 704 at its proximal end. Fresnel lens 704 directs the light rays provided by the illumination system of endoscope 1000. Each groove of fresnel lens 704 may be at a slightly different angle than the next but may utilize the same focal length in order to focus the light directly towards a surgical object or provide a specific field of view for the image transmitting means. Fresnel lens 704 may be constructed of a variety of different materials including, but not limited to glass, acrylic glass, polyvinyl chloride, polycarbonate, or high density polyethylene. The remainder portion of optical transmitting member 702 may be hollow or solid. Fresnel lens 704 may also be utilized as the image receiving member to receive the image as appreciated by one skilled in the art.

It will be understood that various modifications and changes in form and detail may be made to the embodiments of the present disclosure without departing from the spirit and scope of the invention. Therefore, the above description should not be construed as limiting the invention but merely as exemplifications of preferred embodiments thereof. Those skilled in the art will envision other modifications within the scope and spirit of the present invention as defined by the claims appended hereto. Having thus described the invention with the details and particularity required by the patent laws, what is claimed and desired protected is set forth in the appended claims.

What is claimed is:

1. An obturator apparatus for penetration of tissue and for permitting visualization, which comprises:
an elongated shaft having proximal and distal ends, and defining a longitudinal axis; and
an optical transmitting member mounted adjacent the distal end of the elongated shaft, adapted to penetrate tissue, the optical transmitting member comprising a transparent material for permitting passage of light therethrough and having internal surfaces defining a hollow internal chamber and external surfaces adapted to penetrate tissue, the optical transmitting member further being adapted to provide an inclined field of view inclined relative to the longitudinal axis.

2. The obturator apparatus according to claims 1 wherein the elongated shaft includes a longitudinal opening adapted for reception of an endoscope.

3. The obturator apparatus according to claim 1 wherein at least one of the internal and external surfaces of the optical transmitting member is a refracting surface adapted to direct the light along a predetermined path.

4. The obturator apparatus according to claim 3 wherein the optical transmitting member is adapted to direct light in general parallel direction with the inclined field of view.

5. The obturator apparatus according to claim 3 wherein the optical transmitting member is adapted to direct light in general parallel relation to the longitudinal axis of the elongated shaft.

6. The obturator apparatus according to claim 1 wherein the optical transmitting member defines a general pyramidal configuration.

7. The obturator apparatus according to claim 1 wherein the optical transmitting member defines a general conical configuration.

8. The obturator apparatus according to claim 1 wherein the optical transmitting member includes a general jeweled configuration.

9. The obturator apparatus according to claim 1 wherein the optical transmitting member includes a fresnel lens.

10. The obturator apparatus according to claim 1 including a piercing member mounted to the optical transmitting member, the piercing member adapted to pierce tissue.

11. The obturator apparatus according to claim 1 wherein the piercing member is coaxially mounted to the optical transmitting member and defines a piercing end extending from the optical transmitting member.

12. The obturator apparatus according to claim 1 wherein the optical transmitting member comprises glass or an optical polymeric material.

13. The obturator apparatus according to claim 1 including illumination means for providing illuminating light and imaging means for transferring an image of an object.

14. An obturator apparatus, which comprises:
an elongated shaft defining proximal and distal ends, and defining a longitudinal axis;
a transparent member adjacent the distal end thereof, the transparent member including a refracting surface adapted to provide an inclined field of view; and
a piercing member mounted to the transparent member and dimensioned to pierce tissue.

15. The obturator apparatus according to claim 14 wherein the piercing member is mounted to the transparent member in coaxial relation therewith.

16. An optical obturator apparatus for penetration of tissue and for permitting visualization of a surgical object with an endoscope coupled to the obturator apparatus, the endoscope being of the type including an illumination system for delivering illuminating light and an imaging system for detecting and transmitting an illuminated image of the surgical object, the obturator apparatus comprising:
an elongated shaft adapted to at least partially receive the endoscope, and having proximal and distal ends, and defining a longitudinal axis; and
an optical transmitting member mounted adjacent the distal end of the elongated shaft, the optical transmitting member adapted to penetrate tissue and defining a hollow interior chamber, the optical transmitting member comprising a transparent material whereby light rays projected by the illumination system of the inserted endoscope passes through the internal chamber for emission outwardly by the optical transmitting member to permit viewing of the surgical object through the optical transmitting member with the imaging system of the endoscope, the optical transmitting member being adapted to provide a field of view angularly offset relative to the longitudinal axis.
